(19) United States
(12) Patent Application Publication
(10) Pub. No.: US 2026/0256732 A1
(43) Pub. Date: Sep. 3, 2026

(54) COMPOSITION FOR IMPROVING RELAXATION

(71) Applicant: CYTOSOLVE, INC., Cambridge, MA (US)

(72) Inventors: V. A. Shiva AYYADURAI, Cambridge, MA (US); Prabhakar DEONIKAR, Cambridge, MA (US)

(73) Assignee: CYTOSOLVE, INC., Cambridge, MA (US)

(21) Appl. No.: 18/846,764

(22) PCT Filed: Oct. 13, 2023
(86) PCT No.: PCT/US2023/035108

§ 371 Date: Sep. 13, 2024

Related U.S. Application Data
(60) Provisional application No. 63/379,481, filed on Oct. 14, 2022.

(51) Int. Cl. A61K 31/352; A61K 31/122; A61K 31/353; A61K 31/56; A61K 31/585; A61P 25/22

(52) U.S. Cl. CPC A61K 31/352; A61K 31/122; A61K 31/353; A61K 31/56; A61K 31/585; A61P 25/22

(57) ABSTRACT

The present disclosure is related to dietary supplements including compositions that include, one or more agents that increase γ-aminobutyric acid (GABA) binding with GABA-A receptor, increase serotonin concentrations, decrease dopamine concentrations, increase brain derived neurotrophic factor (BDNF), decrease cortisol concentrations, increase anandamide (AEA) concentrations and decrease IL-7 & IL-17 concentrations, responsible for improving relaxation.

### TECHNICAL FIELD

This disclosure is related to the field of dietary supplements. Dietary supplements include compositions useful for improving relaxation or lowering stress. Such compositions may include one or more agents that increase γ-aminobutyric acid (GABA) binding with GABA-A receptor, increase serotonin concentrations, decrease dopamine concentrations, increase brain derived neurotrophic factor (BDNF), decrease cortisol concentrations, increase anandamide (AEA) concentrations and decrease IL-7 & IL-17 concentrations.

### BACKGROUND

Stress is an inevitable part of life, which affects a constellation of physiological systems in the body and evokes a rapid shift in many neurobehavioral and it is an immediate engagement of multiple neural and endocrine systems. However, exposure to a single stressor causes adaptive changes that modify responses to subsequent stressors. Multiple factors influence an individual's ability to cope with stress, for example, early life experiences, gender, or personality traits. Both vulnerability and resilience may be determined by genetic and epigenetic (gene environmental interactions) background.

Stress can be divided into acute stress and chronic stress. Acute stress usually exists in emergencies, such as fighting or escaping. Changes in the structure and function of certain molecules and tissues in the brain activate the emotional cognitive system, and we make decisions for stress-coping mechanisms. At the same time, the body temporarily produces catecholamines and corticosteroids to improve mobility and responsiveness. Therefore, acute stress is often beneficial to the body. Acute stressors such as unexpected and potentially threatening changes of an individual's environment usually provoke immediate stress responses to adapt, which are accompanied by and actuating a number of physiological processes. Stress is the process through which body-brain integration plays a major role and the acute stress in terms of neurotransmitters such as dopamine, serotonin, acetylcholine, glutamate and γ-aminobutyric acid (GABA) in areas of the brain involved in the regulation of stress responses. These areas include the prefrontal cortex, amygdala, hippocampus and nucleus accumbens and the interaction among those areas.

Diet is a significant contributing factor that modulates stress. Various components of the diet including dietary fats, dietary carbohydrates, and micronutrients modulate stress response. Plant-derived micronutrients have been ascribed properties that have been modulate biological processes such as neurotransmitter signaling, endocannabinoid signaling, nerve growth factor signaling, catecholamine synthesis, hypothalamic-pituitary-adrenal (HPA) axis signaling, and gut microbiome neuro-inflammatory signaling that are implicated in stress biology. Methods and compositions that include the exogenous molecules for modulating stress are highly desirable.

### SUMMARY

Provided herein are methods for improving stress response in a subject in need thereof comprising administering to the subject a composition comprising two or more agents that increase γ-aminobutyric acid (GABA) binding with GABA-A receptor, increase serotonin concentrations, decrease dopamine concentrations, increase brain derived neurotrophic factor (BDNF), decrease cortisol concentrations, increase anandamide (AEA) concentrations and decrease IL-6 & IL-17 concentrations.

In some embodiments, one or more agents in combination that decrease the concentration of IL-6; or decreases concentration of IL-17 or a combination thereof are administered.

In some embodiments, one or more agents in combination that decrease the concentration of IL-6; and/or decrease concentration of IL-17 and/orincrease concentration of serotonin or a combination thereof, are administered.

In some embodiments, at least one or more agents in combination that increase GABA binding with GABA-A receptor; and/or decreases cortisol concentrations, and/or increases serotonin concentrations, and/or decreases dopamine concentrations or increases BDNF concentrations or a combination thereof are administered.

In some embodiments, the two or more agents that increase γ-aminobutyric acid (GABA) binding with GABA-A receptor, increase serotonin concentrations, decrease dopamine concentrations, increase brain derived neurotrophic factor (BDNF), decrease cortisol concentrations, increase anandamide (AEA) concentrations and decrease IL-6 & IL-17 concentrations, comprise one or more of: a benzopyrans; an anthracene; a phytosterol; triterpenoid, and, a flavan are administered.

Also provided herein are methods for improving stress in a subject in need thereof comprising administering to the subject a composition comprising two or more agents that increase GABA binding with GABA-A receptor, increase serotonin concentrations, decrease dopamine concentrations, and/or increase BDNF, and/or decrease cortisol concentrations, and/or increase AEA concentrations and/or decrease IL-6 & IL-17 concentrations; or a combination thereof.

In some embodiments, stress is associated with one or more of: cellular toxicity, neuroinflammation, neurodegenerative diseases such as Parkinson's disease, Alzheimer's disease, amyotrophic lateral sclerosis (ALS), multiple sclerosis, depression, and memory loss, anxiety, or aging.

In some of any of the above embodiments, the wherein the benzopyran is a flavonol. In some embodiments, the flavonol is selected from the group consisting of: kaempferol; myricetin; quercetin; fisetin; rhamnazin; and, a combination thereof.

In some embodiments, the flavonol is kaempferol.

In some embodiments, the flavonol is present in an amount of about 0.1% to about 60% w/w of the composition. In some embodiments, the flavonol is present in an amount of about 1% to about 50% w/w of the composition. In some embodiments, the flavonol is present in an amount of about 10% to about 40% w/w of the composition. In some embodiments, the flavonol is present in an amount of about 34.6% w/w of the composition.

In some of any of the above embodiments, the wherein the anthracenes is a naphtodianthrones. In some embodiments, the naphtodianthrones is selected from the group consisting of: hypericin; pseudohypericin; isohypericin; and, a combination thereof.

In some embodiments, the naphtodianthrones is hypericin.

In some embodiments, the naphtodianthrones is present in an amount of about 0.01% to about 60% w/w of the composition. In some embodiments, the naphtodianthrones is present in an amount of about 0.5% to about 50% w/w of the composition. In some embodiments, the naphtodianthrones is present in an amount of about 1% to about 20% w/w of the composition. In some embodiments, the naphtodianthrones is present in an amount of about 1.05% w/w of the composition.

In some of any of the above embodiments, the wherein the phytosterol is a withanolide. In some embodiments, the withanolide is selected from the group consisting of: withanolide A, withanolide B, withaferin A, withanone, and a combination thereof.

In some embodiments, the withanolide is withanone.

In some embodiments, the withanolide is present in an amount of about 0.1% to about 60% w/w of the composition. In some embodiments, the withanolide is present in an amount of about 0.5% to about 40% w/w of the composition. In some embodiments, the withanolide is present in an amount of about 1% to about 15% w/w of the composition. In some embodiments, the withanolide is present in an amount of about 3.35% w/w of the composition.

In some of any of the above embodiments, the wherein the triterpenoid is a pentacyclic triterpenoid. In some embodiments, the pentacyclic triterpenoid is selected from the group consisting of: oleanolic acid; glycyrrhizin; glycyrrhetinic acid; ursolic acid; and, a combination thereof.

In some embodiments, the pentacyclic triterpenoid is ursolic acid.

In some embodiments, the pentacyclic triterpenoid is present in an amount of about 0.1% to about 60% w/w of the composition. In some embodiments, the pentacyclic triterpenoid is present in an amount of about 0.5% to about 50% w/w of the composition. In some embodiments, the pentacyclic triterpenoid is present in an amount of about 5% to about 25% w/w of the composition. In some embodiments, the pentacyclic triterpenoid is present in an amount of about 18.87% w/w of the composition.

In some of any of the above embodiments, the wherein the triterpenoid is a pentacyclic triterpenoid. In some embodiments, the pentacyclic triterpenoid is selected from the group consisting of: oleanolic acid; glycyrrhizin; glycyrrhetinic acid; ursolic acid; and, a combination thereof.

In some embodiments, the pentacyclic triterpenoid is oleanolic acid.

In some embodiments, the pentacyclic triterpenoid is present in an amount of about 0.1% to about 60% w/w of the composition. In some embodiments, the pentacyclic triterpenoid is present in an amount of about 0.5% to about 50% w/w of the composition. In some embodiments, the pentacyclic triterpenoid is present in an amount of about 5% to about 30% w/w of the composition. In some embodiments, the pentacyclic triterpenoid is present in an amount of about 22.00% w/w of the composition.

In some of any of the above embodiments, the wherein the flavan is a flavan-3-ol. In some embodiments, the flavan-3-ol is selected from the group consisting of: catechin; epicatechin gallate; epigallocatechin; epigallocatechin gallate; and, a combination thereof.

In some embodiments, the flavan-3-ol is catechin.

In some embodiments, the flavan-3-ol is present in an amount of about 0.1% to about 60% w/w of the composition. In some embodiments, the flavan-3-ol is present in an amount of about 0.5% to about 50% w/w of the composition. In some embodiments, the flavan-3-ol is present in an amount of about 5% to about 30% w/w of the composition. In some embodiments, the flavan-3-ol is present in an amount of about 20.13% w/w of the composition.

In some embodiments, the benzopyran is kaempferol, the anthracene is hypericin; the phytosterol is withanone; the triterpenoid is ursolic acid and/or oleanic acid; and, the flavan is catechin.

In some embodiments, the composition comprises two or more of: kaempferol; hypericin; withanone; ursolic acid; oleanic acid; and, catechin. In some embodiments, the composition comprises three or more of: kaempferol; hypericin; withanone; ursolic acid; oleanic acid; and, catechin. In some embodiments, the composition comprises four or more of: kaempferol; hypericin; withanone; ursolic acid; oleanic acid; and, catechin. In some embodiments, the composition comprises five or more of: kaempferol; hypericin; withanone; ursolic acid; oleanic acid; and, catechin.

In some embodiments, the composition comprises: kaempferol present in an amount of about 0.1% to about 60% w/w of the composition; hypericin present in an amount of about 0.01% to about 60% w/w of the composition; withanone present in an amount of about 0.1% to about 60% w/w of the composition; ursolic acid present in an amount of about 0.1% to about 60% w/w of the composition; oleanic acid present in an amount of about 0.1% to about 60% w/w of the composition; and, catechin present in an amount of about 0.1% to about 60% w/w of the composition.

In some embodiments, the composition comprises: kaempferol present in an amount of about 34.6% w/w of the composition; hypericin present in an amount of about 1.05% w/w of the composition; withanone present in an amount of about 3.35% w/w of the composition; ursolic acid present in an amount of about 18.87% w/w of the composition; oleanic acid present in an amount of about 22.00% w/w of the composition; and, catechin present in an amount of about 20.13% w/w of the composition.

In some embodiments, the composition further comprises one or more excipients, diluents, or carriers.

In some embodiments, the composition is administered orally.

In some embodiments, the composition is configured as a powder.

Also provided herein is a method for decreasing IL-7 & IL-17 concentrations, decreasing cortisol concentrations, decreasing dopamine concentrations, increasing GABA binding with GABA-A receptor, increasing BDNF concentrations, increasing AEA concentrations, and/or increasing serotonin concentrations of a subject comprising administering to the subject a composition as described herein.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows bar graphs comparing the steady state concentrations of IL-6 for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the IL-6 levels are 1.2×10⁻⁵ nM, whereas for in individuals experiencing stress, the IL-6 levels fell to 4.36×10⁻⁹ nM with supplementation of the entire composition described herein.

FIG. 2 shows bar graphs comparing the steady state concentrations of IL-17 for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the IL-17 levels are 8.57 nM, whereas for in individuals experiencing stress, the IL-17 levels fell to 0.004 nM with supplementation of the entire composition described herein.

FIG. 3 shows bar graphs comparing the steady state concentrations of cortisol for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the cortisol levels are 110 nM, whereas for in individuals experiencing stress, the cortisol levels fell to 94 nM with supplementation of the entire composition described herein.

FIG. 4 shows bar graphs comparing the steady state values of GABA binding to GABA-A receptor for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the GABA binding to GABA-A receptor levels are 224 AUC, whereas for in individuals experiencing stress, GABA binding to GABA-A receptor levels increased to 264 AUC with supplementation of the entire composition described herein.

FIG. 5 shows bar graphs comparing the steady state concentrations of serotonin for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the serotonin levels are 4.26 nM, whereas for in individuals experiencing stress, the serotonin levels increased to 56 nM with supplementation of the entire composition described herein.

FIG. 6 shows bar graphs comparing the steady state concentrations of dopamine for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the dopamine levels are 228 nM, whereas for in individuals experiencing stress, the dopamine levels fell to 123 nM with supplementation of the entire composition described herein.

FIG. 7 shows bar graphs comparing the steady state concentrations of BDNF for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the BDNF levels are 0.71 nM, whereas for in individuals experiencing stress, the BDNF levels increased to 1.25 nM with supplementation of the entire composition described herein.

FIG. 8 shows bar graphs comparing the steady state concentrations of AEA for individuals experiencing stress without supplementation of composition described herein, with individuals experiencing stress with supplementation of the entire composition described herein over a period of 24 hours. The plot is based on a biomolecular computational model using CytoSolve® and modeling mechanisms of stress. The results show that for individuals experiencing stress without supplementation of composition, the AEA levels are 0.006 nM, whereas for in individuals experiencing stress, the AEA levels increased to 0.015 nM with supplementation of the entire composition described herein.

### DETAILED DESCRIPTION

Stress is an inevitable part of life, which affects a constellation of physiological systems in the body and evokes a rapid shift in many neurobehavioral processes and it is an immediate engagement of multiple neural and endocrine systems. However, exposure to a single stressor causes adaptive changes that modify responses to subsequent stressors. Multiple factors influence an individual's ability to cope with stress, for example, early life experiences, gender, or personality traits. Both vulnerability and resilience may be determined by genetic and epigenetic (gene environmental interactions) background. Additionally, Stress exposure also provokes a shift in many neurobehavioral processes, such as anxiety/vigilance, memory, reward salience, pain sensitivity, and coping behaviors. Collectively, these changes in biological function produce coordinated and highly adaptive responses that are conducive to survival in response to a threat. Stress can be either a triggering or aggravating factor for many diseases and pathological conditions.

Diet is a significant contributing factor that modulates systemic stress. Various compounds of the diet including dietary fats, dietary carbohydrates, and micronutrients affect stress. Dietary changes can positively affect stress-related mental disorders, including major depression and posttraumatic stress disorder (PTSD). On the other hand, increased deposition of intraabdominal fat upregulates stress hormone cortisol. High fat diets can lead to leakiness of the gut epithelium, resulting in the release of inflammatory factors and penetration of gut flora in the intestinal wall, which can further increase the risk of stress-induced depression via alterations in signaling pathways leading to the brain Compositions, as described herein, mitigate stress via decreasing the production of stress biomarkers such as IL-6, IL-17, cortisol, dopamine; and, increasing biomarkers such as BDNF, serotonin, GABA binding to GABA-A receptor, and AEA.

Accordingly, the present disclosure provides methods and compositions (e.g., dietary supplements) related to decrease stress. Such compositions can contain two or more agents that reduce the production of IL-6 & IL-17, reduce the production of cortisol, reduce the production of dopamine, increase the production of BDNF, increase the production of serotonin, increase the GABA binding to GABA-A receptor, increase the production of AEA, or a combination thereof, useful for improvement in reduction in stress.

### Definitions

As used herein, the phrase “Stress,” or “low production of IL-6 & IL-17,” or “low production of cortisol,” or “low production of dopamine,” or “high production of BDNF,” or “high binding of GABA to GABA-A receptor,” “high production of BDNF,” “high production of serotonin,” “high production of AEA,” or a disease, disorder, or condition encompasses a subject with high production of IL-6 & IL-17, cortisol, and/or dopamine; and, low production of BDNF, serotonin, and/or AES; and, low binding of GABA with GABA-A receptor, that has also been diagnosed with, was previously diagnosed with, or has symptoms associated with the disease, disorder, or condition.

As used herein, the phrases an “effective amount” or a “therapeutically effective amount” of an active agent or ingredient, or pharmaceutically active agent or ingredient, refer to an amount of the active agent sufficient enough to reduce or eliminate one or more symptoms of the disorder or to effect a cure upon administration. Effective amounts of the active agent will vary with the kind of active agent chosen, the particular condition or conditions being treated, the severity of the condition, the duration of the treatment, the specific components of the composition being used, and like factors.

As used herein, “subject” refers to any subject, particularly a mammalian subject, for whom diagnosis, prognosis, or therapy is desired, for example, a human.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

Agents that Decrease Stress

In some embodiments, a composition as described herein can comprise an agent that decreases production of IL-6 and IL-17 in the gut. For example, in some embodiments, an agent that decreases inflammation is an agent that decrease the production of cortisol and dopamine. As another example, an agent that decreases stress can be an agent that increases production of BDNF, GABA binding to GABA-A receptor, serotonin, and/or AEA. Many agents that decrease the production of IL-6, IL-17, cortisone, and/or dopamine, and increase the production of BDNF, GABA binding to GABA-A receptor, serotonin, and/or AEA are known to one of skill in the art. Non-limiting examples of an agent that decreases the production of production of IL-6, IL-17, cortisone, and/or dopamine; and increases the production of BDNF, GABA binding to GABA-A receptor, serotonin, and/or AEA include kaempferol, hypericin, withanone, ursolic acid, oleanic acid, and catechin. Several methods for measuring IL-6, IL-17, cortisone, dopamine, BDNF, GABA binding to GABA-A receptor, serotonin, and/or AEA are known to one of ordinary skill in the art. Non-limiting examples of such methods include: measuring IL-6 concentration using enzyme-linked immuno-sorbant assay (ELISA) (see, for example, Chen et al., J Immunol Methods. 2016 July; 434:1-8., which is incorporated herein in its entirety), measuring IL-17 concentration using ELISA (see, for example, Pappu and Dong, Curr Protoc Immunol. 2007 November; Chapter 6:6.25.1-6.25.8, which is incorporated herein in its entirety), measuring cortisol concentration using immunoassay or LC-MS/MS (see, for example, Farhan et al., Ann Clin Biochem. 2017 May; 54(3):308-322, which is incorporated herein in its entirety), measuring dopamine concentration using ELISA (see, for example, Nichkova et al., J Neurochem. 2013 June; 125(5):724-735, which is incorporated herein in its entirety), measuring BDNF protein levels serum/blood using immunoprecipitation (e.g., a Western blot) (see, for example, Polacchini et al., Sci. Rep. 2015; 5:17989, which is incorporated herein in its entirety), measuring GABA binding to GABA-A receptor using radioligand displacement assays (see, for example, Elgarf et al., ACS Chem. Biol. 2018; 13, 8, 2033-2039, which is incorporated herein in its entirety), measuring serotonin using platelet serotonin-release assay (see, for example, Warkentin et al., ACS Chem. Biol. 2015 June; 90(6):564-72, which is incorporated herein in its entirety), measuring AEA using LC-MS analysis (see, for example, Buczynski and Parsons, Br J Pharmacol. 2010 June; 160(3): 423-442, which is incorporated herein in its entirety).

In some embodiments, an agent that decrease the concentration of IL-6, IL-17, cortisol, and/or dopamine by at least by about 5%, about 10%, about 25%, about 50%, about 75%, about 99% or more. For example, the agent that decreases the concentration of production of IL-6, IL-17, cortisol, and/or dopamine increases the binding of GABA to GABA-A receptor, concentration of BDNF, serotonin, and/or AEA by about 10%, about 50%, about 75%, about 175%, about 500%, about 1000%, about 1500% or more.

### Benzopyrans

In some embodiments, a composition as described herein includes a benzopyran that reduces production of IL-6. In some embodiments, a composition as described herein includes a benzopyran that reduces production of IL-17. In some embodiments, a composition as described herein includes a benzopyran that increases production of AEA.

As described herein, a “benzopyran” refers to a polycyclic organic compound comprising a benzene ring fused to a pyran ring.

Non-limiting examples of benzopyran include flavonol. Examples of a flavonol include, without limitation, kaempferol, myricetin, quercetin, fisetin, and rhamnazin. Non-limiting examples of benzopyran that can decrease production of IL-6 include kaempferol, quercetin, and myricetin. Non-limiting examples of benzopyran that can decrease production of IL-17 include kaempferol, quercetin, and myricetin. Non-limiting examples of benzopyran that can increase production of AEA include kaempferol, quercetin, and myricetin.

### Anthracene

In some embodiments, a composition as described herein includes an anthracene that reduces concentration levels of cortisol. In some embodiments, a composition as described herein includes an anthracene that reduces production of dopamine. In some embodiments, a composition as described herein includes an anthracene that increases production of BDNF. In some embodiments, a composition as described herein includes an anthracene that increases production of serotonin. In some embodiments, a composition as described herein includes an anthracene that increases binding of GABA to its receptor GABA-A.

As described herein, an “anthracene” refers to a molecule that is a polycyclic aromatic hydrocarbon of formula C₁₄H₁₀, consisting of three fused benzene rings.

Non-limiting examples of anthracene include naphtodianthrones. Examples of a naphtodianthrones include, without limitation, hypericin, pseudohypericin, and isohypericin. Non-limiting examples of naphtodianthrones that reduce production of cortisol include hypericin, pseudohypericin, and isohypericin. Non-limiting examples of naphtodianthrones that reduce production of dopamine include hypericin, pseudohypericin, and isohypericin. Non-limiting examples of naphtodianthrones that increase production of serotonin include hypericin, pseudohypericin, and isohypericin. Non-limiting examples of naphtodianthrones that increase production of BDNF include hypericin, pseudohypericin, and isohypericin. Non-limiting examples of naphtodianthrones that increase binding of GABA to its receptor GABA-A include hypericin, pseudohypericin, and isohypericin.

### Phytosterol

In some embodiments, a composition as described herein includes a phytosterol that reduces production of IL-6. In some embodiments, a composition as described herein includes a phytosterol that reduces production of IL-17. In some embodiments, a composition as described herein includes a phytosterol that increases production of serotonin.

As described herein, a “phytosterol” refers to a molecule that has a polycyclic structure and vary in carbon side chains and/or presence or absence of a double bond and are divided into 4,4-dimethyl phytosterols, 4-monomethyl phytosterols, and 4-desmethyl phytosterols based on the location of methyl groups at the carbon-4 position.

Non-limiting examples of phytosterol include withanolides. Examples of a withanolides include, without limitation, withanolide A, withanolide B, withaferin A, and withanone. Non-limiting examples of withanolides that can decrease production of IL-6 include withanolide A, withanolide B, withaferin A, and withanone. Non-limiting examples of withanolides that can decrease production of IL-17 include withanolide A, withanolide B, withaferin A, and withanone. Non-limiting examples of withanolides that can increase production of serotonin include withanolide A, withanolide B, withaferin A, and withanone.

### Triterpinoids

In some embodiments, a composition as described herein includes a triterpinoid that reduces production of IL-6. In some embodiments, a composition as described herein includes a triterpinoids that reduces production of IL-17.

As described herein, a “triterpenoid” refers to a molecule that has six isoprene units with the molecular formula C₃₀H₄₈.

Non-limiting examples of phytosterol include pentacyclic triterpenoid. Examples of a triterpinoids include, without limitation, oleanolic acid, glycyrrhizin, glycyrrhetinic acid, and ursolic acid. Non-limiting examples of triterpinoids that can decrease production of IL-6 include oleanolic acid, glycyrrhizin, glycyrrhetinic acid, and ursolic acid. Non-limiting examples of triterpenoid that can decrease production of IL-17 include oleanolic acid, glycyrrhizin, glycyrrhetinic acid, and ursolic acid.

### Flavan

In some embodiments, a composition as described herein includes a flavan that reduces concentration levels of IL-6 and IL-17.

As described herein, a “flavan” refers to a molecule that have the 3-hydroxy-2-phenylchromen-4-one skeleton with different positions of the phenolic-OH groups.

Non-limiting examples of flavan include flavan-3-ols, flavan-4-ols and flavan-3,4-diols. Examples of a flavan-3-ols include, without limitation, epicatechin, catechin, epigallocatechin, fisetinidol, and EGCG. Non-limiting examples of flavans that reduces production of IL-6 include epicatechin and EGCG. Non-limiting examples of flavans that reduces production of IL-17 include epicatechin and EGCG.

### Dietary Supplemental Compositions

The present disclosure provides compositions (e.g., dietary supplements) containing two or more agents that increase GABA binding with GABA-A receptor, increase serotonin concentrations, decrease dopamine concentrations, increase BDNF concentrations, decrease cortisol concentrations, increase AEA concentrations and decrease IL-6 & IL-17 concentrations. For example, in some embodiments, the present disclosure provides a compositions comprising two or more agents that increase GABA binding with GABA-A receptor. As another example, in some embodiments, the present disclosure provides a composition comprising two or more agents that increase GABA binding with GABA-A receptor; increase serotonin concentrations; or a combination thereof. As another example, in some embodiments, the present disclosure provides a composition comprising two or more agents that increase GABA binding with GABA-A receptor; increase serotonin concentrations; decrease dopamine concentrations; or a combination thereof. As another example, in some embodiments, the present disclosure provides a composition comprising two or more agents that increase GABA binding with GABA-A receptor; increase serotonin concentrations; decrease dopamine concentrations; increase BDNF concentrations; or a combination thereof. As another example, in some embodiments, the present disclosure provides a composition comprising two or more agents that increase GABA binding with GABA-A receptor; increase serotonin concentrations; decrease dopamine concentrations; increase BDNF concentrations; decrease cortisol concentrations; or a combination thereof. As another example, in some embodiments, the present disclosure provides a composition comprising two or more agents that increase GABA binding with GABA-A receptor; increase serotonin concentrations; decrease dopamine concentrations; increase BDNF concentrations; decrease cortisol concentrations; increase AEA concentrations or a combination thereof. As another example, in some embodiments, the present disclosure provides a composition comprising two or more agents that increase GABA binding with GABA-A receptor; increase serotonin concentrations; decrease dopamine concentrations; increase BDNF concentrations; decrease cortisol concentrations; increase AEA concentrations; decrease IL-6 & IL-17 concentrations or a combination thereof. In some embodiments, the present disclosure provides compositions comprising a benzopyrans, an anthracene; a phytosterol; triterpenoid; and, a flavan. Such compositions can be used to decrease stress. In addition, the disclosure provides methods for relieving symptoms caused by stress associated with one or more of: cellular toxicity, neuroinflammation, neurodegenerative diseases such as Parkinson's disease, Alzheimer's disease, amyotrophic lateral sclerosis (ALS), multiple sclerosis, depression, and memory loss, anxiety, or aging. Such methods involve the administration of a composition as provided herein.

In some embodiments, the two or more agents that increase γ-aminobutyric acid (GABA) binding with GABA-A receptor, increase serotonin concentrations, decrease dopamine concentrations, increase brain derived neurotrophic factor (BDNF), decrease cortisol concentrations, increase anandamide (AEA) concentrations and decrease IL-7 & IL-17 concentrations, comprise one or more of: a benzopyrans; an anthracene; a phytosterol; triterpenoid; and, a flavan.

Also provided herein are methods for improving stress in a subject in need thereof comprising administering to the subject a composition comprising two or more agents that increase GABA binding with GABA-A receptor, increase serotonin concentrations, decrease dopamine concentrations, increase BDNF, decrease cortisol concentrations, increase AEA concentrations and decrease IL-6 & IL-17 concentrations; or a combination thereof.

In some embodiments, stress is associated with one or more of: cellular toxicity, neuroinflammation, neurodegenerative diseases such as Parkinson's disease, Alzheimer's disease, amyotrophic lateral sclerosis (ALS), multiple sclerosis, depression, and memory loss, anxiety, or aging.

In some embodiments, a composition as described herein includes a benzopyran that reduces stress. In some embodiments, a composition as described herein includes a benzopyran that reduces production of IL-6. In some embodiments, a composition as described herein includes a benzopyran that reduces production of IL-17. In some embodiments, a composition as described herein includes a benzopyran that increases production of AEA.

As described herein, a “benzopyran” refers to a polycyclic organic compound comprising a benzene ring fused to a pyran ring. Non-limiting examples of benzopyran include flavonol. Examples of a flavonol include, without limitation, kaempferol, myricetin, quercetin, fisetin, and rhamnazin. Non-limiting examples of benzopyran that can decrease production of IL-6 include kaempferol, quercetin, and myricetin. Non-limiting examples of benzopyran that can decrease production of IL-17 include kaempferol, quercetin, and myricetin. Non-limiting examples of benzopyran that can increase production of AEA include kaempferol, quercetin, and myricetin.

In some embodiments, a benzopyran is present in an amount of about 0.1% to about 60% w/w of the composition. For example, about 0.1% to about 10%, about 0.1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 40% to about 60%, and about 10% to about 60% of the composition. In some embodiments, a benzopyran is present in an amount of about 1% to about 50% w/w of the composition. For example, about 1% to about 10%, about 1% to about 20%, about 1% to about 30%, about 1% to about 40%, about 1% to about 49%, about 49% to about 50%, about 40% to about 50%, about 30% to about 50%, about 20% to about 50%, and about 10% to about 50% of the composition. In some embodiments, a benzopyran is present in an amount of about 5%, about 10%, about 20%, about 30%, about 32%, about 33%, about 34.2%, about 34.4%, about 34.5%, about 34.6, about 34.8%, about 40%, about 40%, about 49.5%, or about 50% w/w of the composition.

In some embodiments, the benzopyran is a flavonol. In some embodiments, the flavonol is selected from the group consisting of: kaempferol, myricetin, quercetin, fisetin, and rhamnazin, and a combination thereof.

In some embodiments, the benzopyran is kaempferol. In some embodiments, kaempferol is present in an amount of about 0.1% to about 60% w/w of the composition. For example, about 0.1% to about 10%, about 0.1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 40% to about 60%, and about 10% to about 60% of the composition. In some embodiments, a kaempferol is present in an amount of about 1% to about 50% w/w of the composition. For example, about 1% to about 10%, about 1% to about 20%, about 1% to about 30%, about 1% to about 40%, about 1% to about 49%, about 49% to about 50%, about 40% to about 50%, about 30% to about 50%, about 20% to about 50%, and about 10% to about 50% of the composition. In some embodiments, a kaempferol is present in an amount of about 5%, about 10%, about 20%, about 30%, about 32%, about 33%, about 34.2%, about 34.4%, about 34.5%, about 34.6, about 34.8%, about 40%, about 40%, about 49.5%, or about 50% w/w of the composition.

In some embodiments, a composition as described herein includes a anthracene that reduces stress. In some embodiments, a composition as described herein includes a anthracene that reduces production of cortisol. In some embodiments, a composition as described herein includes a anthracene that increases the production of BDNF. In some embodiments, a composition as described herein includes a anthracene that increases production of GABA binding to GABA-A receptor.

As described herein, an “anthracene” refers to a molecule that is a polycyclic aromatic hydrocarbon of formula C₁₄H₁₀, consisting of three fused benzene rings. Examples of a naphtodianthrones include, without limitation, hypericin, pseudohypericin, and isohypericin. Non-limiting examples of naphtodianthrones that reduce production of cortisol include hypericin, pseudohypericin, and isohypericin. Non-limiting examples of naphtodianthrones that reduce production of dopamine include hypericin, pseudohypericin, and isohypericin. Non-limiting examples of naphtodianthrones that increase production of serotonin include hypericin, pseudohypericin, and isohypericin. Non-limiting examples of naphtodianthrones that increase production of BDNF include hypericin, pseudohypericin, and isohypericin. Non-limiting examples of naphtodianthrones that increase binding of GABA to its receptor GABA-A include hypericin, pseudohypericin, and isohypericin.

In some embodiments, an anthracene is present in an amount of about 0.01% to about 60% w/w of the composition. For example, about 0.01% to about 10%, 0.01% to about 20%, about 0.01% to about 30%, about 0.01% to about 40%, about 0.01% to about 50%, about 0.01% to about 59%, about 59% to about 60%, about 50% to about 60%, about 40% to about 60%, about30% to about 60%, about 20% to about 60%, and about 10% to about 60% w/w of the composition. In some embodiments, an anthracene is present in an amount of about 1% to about 20% w/w of the composition. For example, about 1% to about 2%, about 1% to about 5%, about 1% to about 10%, about 1% to about 15%, about 1% to about 19%, about 19% to about 20%, about 15% to about 20%, about 10% to about 20%, about 5% to about 20%, or about 2% to about 20%. In some embodiments, an anthracene is present in an amount of about 0.1%, about 0.5%, about 0.75%, about 0.85%, about 0.95%, about 1.0%, about 1.01%, about 1.02%, about 1.03%, about 1.04%, about 1.05%, about 2%, about 5%, about 10%, or about 20% w/w of the composition.

In some embodiments, the anthracene is a naphtodianthrones. In some embodiments, the naphtodianthrones is selected from the group consisting of: hypericin, pseudohypericin, and isohypericin; and, a combination thereof.

In some embodiments, the anthracene is hypericin. In some embodiments, hypericin is present in an amount of about 0.01% to about 60% w/w of the composition. For example, about 0.01% to about 10%, 0.01% to about 20%, about 0.01% to about 30%, about 0.01% to about 40%, about 0.01% to about 50%, about 0.01% to about 59%, about 59% to about 60%, about 50% to about 60%, about 40% to about 60%, about30% to about 60%, about 20% to about 60%, and about 10% to about 60% w/w of the composition. In some embodiments, a hypericin is present in an amount of about 1% to about 20% w/w of the composition. For example, about 1% to about 2%, about 1% to about 5%, about 1% to about 10%, about 1% to about 15%, about 1% to about 19%, about 19% to about 20%, about 15% to about 20%, about 10% to about 20%, about 5% to about 20%, or about 2% to about 20%. In some embodiments, a hypericin is present in an amount of about 0, 1%, about 0.5%, about 0.75%, about 0.85%, about 0.95%, about 1.0%, about 1.01%, about 1.02%, about 1.03%, about 1.04%, about 1.05%, about 2%, about 5%, about 10%, or about 20% w/w of the composition.

In some embodiments, a composition as described herein includes a phytosterol that reduces stress. In some embodiments, a composition as described herein includes a phytosterol that reduces production of IL-6. In some embodiments, a composition as described herein includes a phytosterol that reduces production of IL-17. In some embodiments, a composition as described herein includes a phytosterol that increases production of serotonin.

As described herein, a “phytosterol” refers to a molecule that has a polycyclic structure and vary in carbon side chains and/or presence or absence of a double bond and are divided into 4,4-dimethyl phytosterols, 4-monomethyl phytosterols, and 4-desmethyl phytosterols based on the location of methyl groups at the carbon-4 position. Non-limiting examples of phytosterol include withanolides. Examples of a withanolides include, without limitation, withanolide A, withanolide B, withaferin A, and withanone. Non-limiting examples of withanolides that can decrease production of IL-6 include withanolide A, withanolide B, withaferin A, and withanone. Non-limiting examples of withanolides that can decrease production of IL-17 include withanolide A, withanolide B, withaferin A, and withanone. Non-limiting examples of withanolides that can increase production of serotonin include withanolide A, withanolide B, withaferin A, and withanone.

In some embodiments, phytosterol is present in an amount of about 0.1% to about 60% w/w of the composition. For example, about 0.1% to about 1%, about 0.1% to about 5%, about 0.1% to about 10%, about 0.1% to about 20%, about 0.1% to about 30%, about 0.1% to about 40%, 0.1% to about 59%, about 59% to about 60%, about 40% to about 60%, about 30% to about 60%, about 20% to about 60%, about 10% to about 60%, about 5% to about 60%, or about 1% to about 60% w/w of the composition. In some embodiments, phytosterol is present in an amount of about 1% to about 15% w/w of the composition. For example, about 1% to about 2%, about 1% to about 5%, about 1% to about 10%, about 1% to about 14.9%, about 14.9% to about 15%, about 10% to about 15%, about 5% to about 15%, or about 2% to about 15% w/w of the composition. In some embodiments, a phytosterol is present in an amount of about 0.1%, about 0.5%, about 1%, about 2%, about 2.5%, about 3%, about 3.1%, about 3.2%, about 3.32%, about 3.34%, about 3.35%, about 3.4%, about 5%, about 10%, or about 15% w/w of the composition.

In some embodiments, the phytosterol is a withanolides. In some embodiments, the withanolides is selected from the group consisting of: proneurosporene, lycopersene, lycopene, phytoene, and a combination thereof.

In some embodiments, the phytosterol is withanone. In some embodiments, withanone is present in an amount of about 0.1% to about 60% w/w of the composition. For example, about 0.1% to about 1%, about 0.1% to about 5%, about 0.1% to about 10%, about 0.1% to about 20%, about 0.1% to about 30%, about 0.1% to about 40%, 0.1% to about 59%, about 59% to about 60%, about 40% to about 60%, about 30% to about 60%, about 20% to about 60%, about 10% to about 60%, about 5% to about 60%, or about 1% to about 60% w/w of the composition. In some embodiments, withanone is present in an amount of about 1% to about 15% w/w of the composition. For example, about 1% to about 2%, about 1% to about 5%, about 1% to about 10%, about 1% to about 14.9%, about 14.9% to about 15%, about 10% to about 15%, about 5% to about 15%, or about 2% to about 15% w/w of the composition. In some embodiments, a withanone is present in an amount of about 0.1%, about 0.5%, about 1%, about 2%, about 2.5%, about 3%, about 3.1%, about 3.2%, about 3.32%, about 3.34%, about 3.35%, about 3.4%, about 5%, about 10%, or about 15% w/w of the composition.

In some embodiments, a composition as described herein includes a triterpinoid that can modulate stress. In some embodiments, a composition as described herein includes a triterpinoid that decreases production of IL-6. In some embodiments, a composition as described herein includes a flavone that decreases production of IL-17.

As described herein, a “triterpinoid” refers to a molecule that has six isoprene units with the molecular formula C₃₀H₄₈. Non-limiting examples of phytosterol include pentacyclic triterpenoid. Examples of a triterpinoids include, without limitation, oleanolic acid, glycyrrhizin, glycyrrhetinic acid, and ursolic acid. Non-limiting examples of triterpinoids that can decrease production of IL-6 include oleanolic acid, glycyrrhizin, glycyrrhetinic acid, and ursolic acid. Non-limiting examples of triterpenoid that can decrease production of IL-17 include oleanolic acid, glycyrrhizin, glycyrrhetinic acid, and ursolic acid.

In some embodiments, a triterpinoid is present in an amount of about 0.1% to about 60% w/w of the composition. For example, about 0.1% to about 10%, about 0, 1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 20% to about 60%, or about 10% to about 60% w/w of the composition. In some embodiments, the triterpinoid is present in an amount of about 5% to about 25% w/w of the composition. For example, about 5% to about 10%, about 5% to about 15%, about 5% to about 20%, about 5% to about 24.9%, about 24.9% to about 25%, about 20% to about 25%, about 15% to about 25%, or about 10% to about 25% w/w of the composition. In some embodiments, the triterpinoid is present in an amount of about 1%, about 5%, about 10%, about 15%, about 17%, about 18%, about 18.2%, about 18.4%, about 18.8%, about 18.84%, about 18.86%, about 18.87%, about 18.9%, about 20%, about 22%, about 23%, about 24%, or about 25% w/w of the composition.

In some embodiments, the triterpinoid is a pentacyclic triterpenoid. In some embodiments, the pentacyclic triterpenoid is selected from the group consisting of: oleanolic acid, glycyrrhizin, glycyrrhetinic acid, ursolic acid, and a combination thereof.

In some embodiments, the triterpinoid is ursolic acid. In some embodiments, ursolic acid is present in an amount of about 00.1% to about 60% w/w of the composition. For example, about 0.1% to about 10%, about 0, 1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 20% to about 60%, or about 10% to about 60% w/w of the composition. In some embodiments, the ursolic acid is present in an amount of about 5% to about 25% w/w of the composition. For example, about 5% to about 10%, about 5% to about 15%, about 5% to about 20%, about 5% to about 24.9%, about 24.9% to about 25%, about 20% to about 25%, about 15% to about 25%, or about 10% to about 25% w/w of the composition. In some embodiments, the ursolic acid is present in an amount of about 1%, about 5%, about 10%, about 15%, about 17%, about 18%, about 18.2%, about 18.4%, about 18.8%, about 18.84%, about 18.86%, about 18.87%, about 18.9%, about 20%, about 22%, about 23%, about 24%, or about 25% w/w of the composition.

In some embodiments, the triterpinoid is oleanolic acid. In some embodiments, oleanolic acid is present in an amount of about 00.1% to about 60% w/w of the composition. For example, about 0.1% to about 10%, about 0, 1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 20% to about 60%, or about 10% to about 60% w/w of the composition. In some embodiments, the oleanolic acid is present in an amount of about 5% to about 25% w/w of the composition. For example, about 5% to about 10%, about 5% to about 15%, about 5% to about 20%, about 5% to about 24.9%, about 24.9% to about 25%, about 20% to about 25%, about 15% to about 25%, or about 10% to about 25% w/w of the composition. In some embodiments, the oleanolic acid is present in an amount of about 1%, about 5%, about 10%, about 15%, about 17%, about 18%, about 19%, about 20%, about 21%, about 22%, about 18.86%, about 18.87%, about 18.9%, about 20%, about 22%, about 23%, about 24%, or about 25% w/w of the composition.

In some embodiments, a composition as described herein includes a flavan that can modulate stress. In some embodiments, a composition as described herein includes a flavan that decreases production of IL-6. In some embodiments, a composition as described herein includes a flavone that decreases production of IL-17.

As described herein, a “flavan” refers to a molecule that have the 3-hydroxy-2-phenylchromen-4-one skeleton with different positions of the phenolic-OH groups. Non-limiting examples of flavan include flavan-3-ols, flavan-4-ols and flavan-3,4-diols. Examples of a flavan-3-ols include, without limitation, epicatechin, catechin, epigallocatechin, fisetinidol, EGCG. Non-limiting examples of flavans that reduces production of IL-6 include epicatechin and EGCG. Non-limiting examples of flavans that reduces production of IL-17 include epicatechin and EGCG.

In some embodiments, a flavan is present in an amount of about 0.1% to about 60% w/w of the composition. For example, about 0.1% to about 10%, about 0, 1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 20% to about 60%, or about 10% to about 60% w/w of the composition. In some embodiments, the flavan is present in an amount of about 5% to about 25% w/w of the composition. For example, about 5% to about 10%, about 5% to about 15%, about 5% to about 20%, about 5% to about 24.9%, about 24.9% to about 25%, about 20% to about 25%, about 15% to about 25%, or about 10% to about 25% w/w of the composition. In some embodiments, the flavan is present in an amount of about 1%, about 5%, about 10%, about 15%, about 17%, about 18%, about 19%, about 20%, about 20.05%, about 20.10%, about 20.11%, about 20.12%, about 20.13%, about 20.15%, about 21%, about 22%, about 23%, about 24%, or about 25% w/w of the composition.

In some embodiments, the flavan is a flavan-3-ol. In some embodiments, the flavan-3-ol is selected from the group consisting of: epicatechin, catechin, epigallocatechin, fisetinidol, EGCG, and a combination thereof.

In some embodiments, the flavan is catechin. In some embodiments, catechin is present in an amount of about 0.1% to about 60% w/w of the composition. For example, about 0.1% to about 10%, about 0, 1% to about 20%, about 0.1% to about 30%, about 0.1% to about 40%, about 0.1% to about 50%, about 0.1% to about 59%, about 59% to about 60%, about 50% to about 60%, about 40% to about 60%, about 30% to about 60%, about 20% to about 60%, or about 10% to about 60% w/w of the composition. In some embodiments, the catechin is present in an amount of about 5% to about 25% w/w of the composition. For example, about 5% to about 10%, about 5% to about 15%, about 5% to about 20%, about 5% to about 24.9%, about 24.9% to about 25%, about 20% to about 25%, about 15% to about 25%, or about 10% to about 25% w/w of the composition. In some embodiments, the catechin is present in an amount of about 1%, about 5%, about 10%, about 15%, about 17%, about 18%, about 19%, about 20%, about 20.05%, about 20.10%, about 20.11%, about 20.12%, about 20.13%, about 20.15%, about 21%, about 22%, about 23%, about 24%, or about 25% w/w of the composition.

In some of any of the above embodiments, the composition further comprises one or more excipients, diluents, or carriers.

In some embodiments, a composition as described herein is formulated for oral delivery. A composition as described herein can be formulated for oral delivery in a variety of ways. For example, the composition can be in the form of a tablet or powder. As another example, a composition as described herein can be in the form of a liquid, solution, suspension, gummy, tablet, powder, soft gelatin capsules, or hard gelatin capsules. Commercial dietary supplements are generally formulated for oral administration. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. For example, a composition as described herein can be presented as dry powder and dissolved in a suitable liquid carrier. In some embodiments, a composition as described herein can be diluted in a suitable liquid carrier. In some embodiments, a composition as described herein is diluted in an energy drink. In some embodiments, liquid preparations also can contain pharmaceutically acceptable additives such as suspending agents, emulsifying agents, non-aqueous vehicles, preservatives, buffer salts, flavoring agents, coloring agents, and sweetening agents as appropriate. In some embodiments, a composition as described herein can be presented as a stick pack. Preparations for oral administration can be suitably formulated to give controlled release of the compound.

Tablets and powders can be configured to have a unit dosage equal to the daily desired dosage. For example, if a subject desires 1000 mg of a particular composition, each tablet can be 1000 mg in weight. As another example, if a subject desires 1000 mg of a particular composition each tablet can be 500 mg in weight and the subject can take two tablets. The dosages of a particular composition will depend on many factors including the mode of administration. As an example, a composition as described herein can be formulated in a dose such that an individual receives the weight percentages as shown in Table 1, e.g., in a single tablet, divided among 2 or more tablets, or as a powder.

TABLE 1

| Components | Weight Percentage (%) |
|---|---|
| Kaempferol | 34.6% |
| Hypericin | 1.05% |
| Withanone | 3.35% |
| Ursolic acid | 18.87% |
| Oleanic acid | 22.00% |
| Catechin | 20.13% |

In addition, a composition provided herein can contain a pharmaceutically acceptable carrier for in vivo administration to a subject. Such pharmaceutically acceptable carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include water, alcohol, saline, and buffered solutions. Pharmaceutically acceptable carriers also can include physiologically acceptable aqueous vehicles (e.g., physiological saline) or other known carriers appropriate to specific routes of administration. Preservatives, flavorings, and other additives such as, for example, proteins, anti-microbials, chelating agents, inert gases, and the like also can be present in a composition.

### Methods

Also provided herein are methods for decreasing stress in a subject in need thereof comprising administering to the subject any of the compositions as described herein. Also provided herein are methods for decreasing stress levels in a subject in need thereof comprising administering to the subject any of the compositions as described herein.

In some embodiments, stress is associated with one or more of: cellular toxicity, infection, cancer, cardiovascular diseases such as atherosclerosis, neurodegenerative diseases such as Parkinson's disease, Alzheimer's disease, amyotrophic lateral sclerosis (ALS), multiple sclerosis, depression, and memory loss, obesity, osteoarthritis, mood disorders, diabetes mellitus, or aging.

In some embodiments, the composition is administered orally.

### EXAMPLES

### Example 1. In Silico Simulations of Compositions for Reducing Stress

### Protocol

The in silico simulations were performed using CytoSolve®, a commercially available tool that enables the computational modeling of biomolecular pathways. CytoSolve® can scale and model highly complex biomolecular phenomena by its ability to integrate and couple the computations of smaller biomolecular pathways (see, e.g., Ayyadurai and Deonikar Nutrients, 2023, Ayyadurai and Deonikar, Appl Sci., 2022, Ayyadurai et al. Clin. Nutr. ESPEN, 2022, Ayyadurai et al. Cancers, 2022, 14, 756; Ayydurai and Deonikar, Clin. Nutr. ESPEN, 2021, 46, pp. 439-452; Ayyadurai and Forbes-Dewey Jr. Cellular and Molecular Bioengineering. 2011, 4(1):28-45; Nordsletten. IEEE Trans Biomed Eng. 2011; 58(12):3508-12; Ayyadurai and Deonikar. Agricultural Sciences. 2015; 6:630-662; Ayyadurai. Commun Med Care Compunetics. 2011; 1:115-168; Koo et al. Biophys J. 2013; 104(10):2295-306; Sweeney et al. Nat Neurosci. 2016; 19(6):771-83; and Ayyadurai (2007) Scalable Computational Architecture for Integrating Biological Pathway Models (Doctoral Dissertation, Massachusetts Institute of Technology); each of which is hereby incorporated by reference in its entirety).

CytoSolve® platform was used to model mechanisms of stress that included production of GABA binding with GABA-A receptor; serotonin; dopamine; BDNF; cortisol; AEA; IL-6; and, IL-17. Once these pathways were integrated using CytoSolve®, the resulting biomolecular computational model was used to identify the ranges of concentrations of kaempferol; hypericin; withanone; ursolic acid; oleanic acid; and, catechin, which elicit a synergistic effect on the biomarkers, GABA binding with GABA-A receptor; serotonin; dopamine; BDNF; cortisol; AEA; IL-6; and, IL-17 (see Table 1). Three scenarios were simulated biomolecular computational model on the CytoSolve® platform over a period of about one (1) day: 1) Control (GABA binding with GABA-A receptor; serotonin; dopamine; BDNF; cortisol; AEA; IL-6; and, IL-17 concentrations without the supplementation of the composition described herein); and, 2) Effect of composition herein in Table 1 on GABA binding with GABA-A receptor; serotonin; dopamine; BDNF; cortisol; AEA; IL-6; and, IL-17 concentration over a period of 1 day. The amounts of kaempferol; hypericin; withanone; ursolic acid; oleanic acid; and, catechin, from Table 1 were used to model the individual as well as synergistic effect on GABA binding with GABA-A receptor; serotonin; dopamine; BDNF; cortisol; AEA; IL-6; and, IL-17 concentration versus the control over a period of one (1) day of supplementation.

### Results

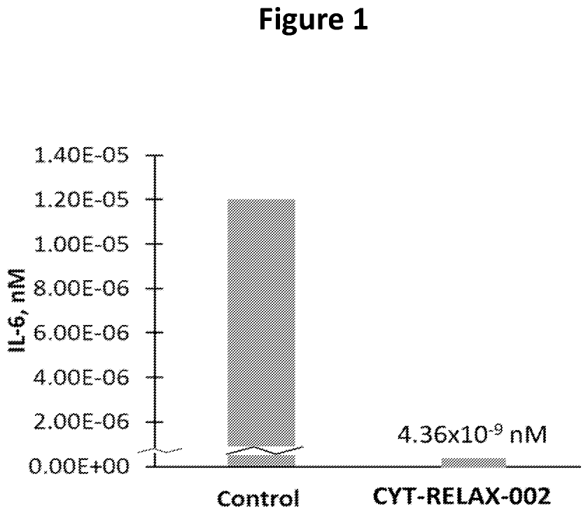

FIG. 1 was derived using CytoSolve® to model mechanisms of stress. The results in FIG. 1 represent steady state concentrations of IL-6 for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the IL-6 levels are 1.2×10⁻⁵ nM. For in individuals experiencing stress with supplementation of the entire composition described herein, the IL-6 levels fell to 4.36×10⁻⁹ nM. These results show that the combination of ingredients from Table 1 is able to reduce the IL-6 levels significantly.

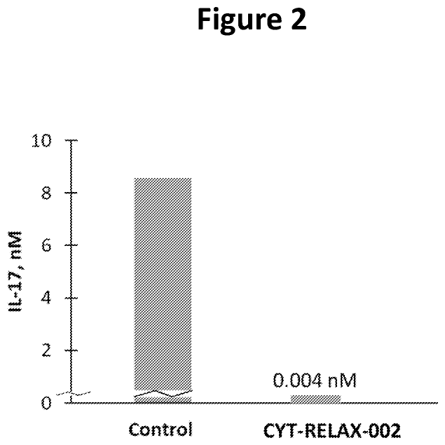

FIG. 2 was derived using CytoSolve® to model mechanisms of stress. The results in FIG. 2 represent steady state concentrations of IL-17 for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the IL-17 levels are 8.57 nM. For in individuals experiencing stress with supplementation of the entire composition described herein, the IL-17 levels fell to 0.004 nM. These results show that the combination of ingredients from Table 1 is able to reduce the IL-17 levels significantly.

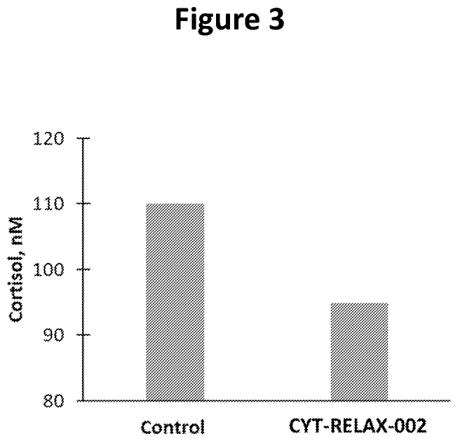

FIG. 3 was derived using CytoSolve® to model mechanisms of stress. The results in FIG. 3 represent steady state concentrations of cortisol for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the cortisol levels are 110 nM. For in individuals experiencing stress with supplementation of the entire composition described herein, the cortisol levels fell to 94 nM. These results show that the combination of ingredients from Table 1 is able to reduce the cortisol levels significantly.

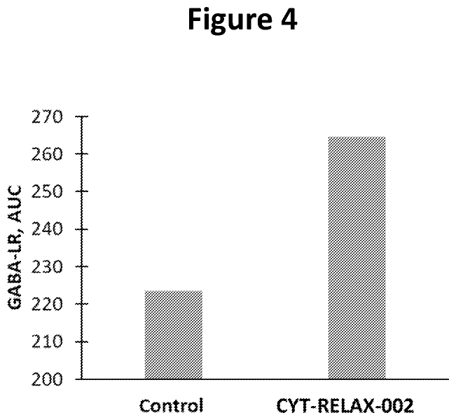

FIG. 4 was derived using CytoSolve® to model mechanisms of stress. The results in FIG. 4 represent steady state concentrations of GABA binding to GABA-A receptor for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the GABA binding to GABA-A receptor levels are 224 AUC. For in individuals experiencing stress with supplementation of the entire composition described herein, the GABA binding to GABA-A receptor levels increased to 264 AUC. These results show that the combination of ingredients from Table 1 is able to increase the GABA binding to GABA-A receptor significantly.

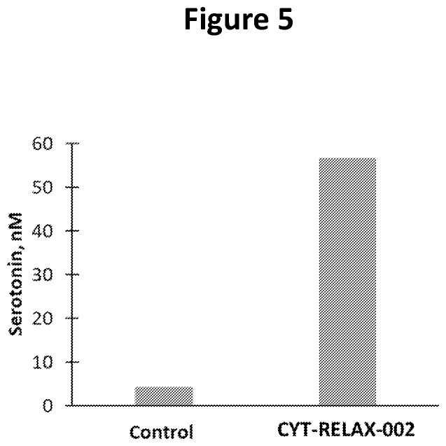

FIG. 5 was derived using CytoSolve® to model mechanisms of stress. The results in FIG. 5 represent steady state concentrations of serotonin for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the serotonin levels are 4.26 nM. For in individuals experiencing stress with supplementation of the entire composition described herein, the serotonin levels increased to 56 nM. These results show that the combination of ingredients from Table 1 is able to increase the serotonin levels significantly.

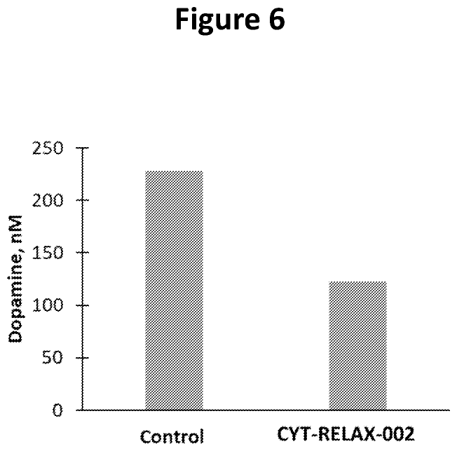

FIG. 6 was derived using CytoSolve® to model mechanisms of stress. The results in FIG. 6 represent steady state concentrations of dopamine for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the dopamine levels are 228 nM. For in individuals experiencing stress with supplementation of the entire composition described herein, the dopamine levels decreased to 123 nM. These results show that the combination of ingredients from Table 1 is able to decease the dopamine levels significantly.

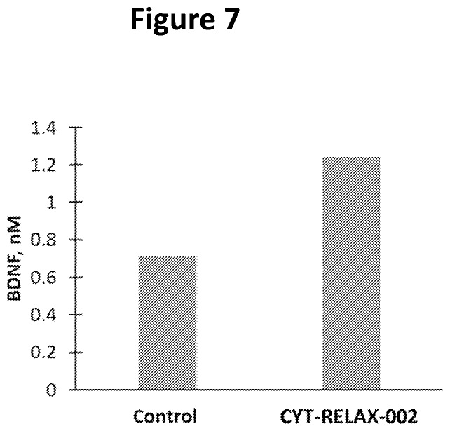

FIG. 7 was derived using CytoSolve® to model mechanisms of stress. The results in FIG. 7 represent steady state concentrations of BDNF for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the BDNF levels are 0.71 nM. For in individuals experiencing stress with supplementation of the entire composition described herein, the BDNF levels increased to 1.25 nM. These results show that the combination of ingredients from Table 1 is able to increase the BDNF levels significantly.

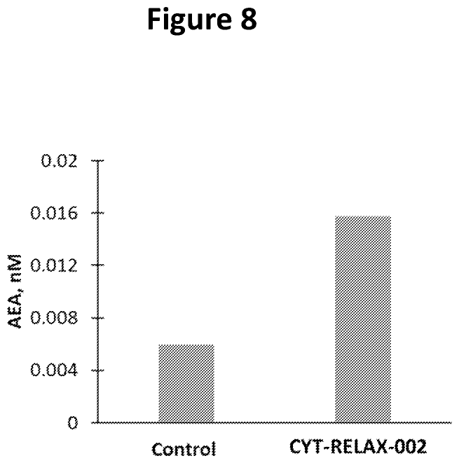

FIG. 8 was derived using CytoSolve® to model mechanisms of stress. The results in FIG. 8 represent steady state concentrations of AEA for individuals experiencing stress without supplementation of composition described herein and with individuals experiencing stress with supplementation of the entire composition described herein over a period of one (1) day. The plot is based on a biomolecular computational model using CytoSolve® and modeling mechanisms of stress. The results show that for individuals experiencing stress without supplementation of composition, the AEA levels are 0.006 nM. For in individuals experiencing stress with supplementation of the entire composition described herein, the AEA levels increased to 0.015 nM. These results show that the combination of ingredients from Table 1 is able to increase the AEA levels significantly.

### Example 2. In Vitro Efficacy Testing of Compositions for Reducing Stress

The in vitro efficacy testing of compositions for reducing production of GABA binding with GABA-A receptor, serotonin, dopamine, BDNF, cortisol, AEA, IL-6, and IL-17 to mitigate stress are being conducted using cell culture model as described in Subash-Babu et al., 2022 (see, e.g., Subash-Babu et al. Molecules 23:4, which is incorporated by reference herein in its entirety). Experiments are being conducted with and without the application of the composition described herein. GABA binding with GABA-A receptor, serotonin, dopamine, BDNF, cortisol, AEA, IL-6, and IL-17 concentrations will be measured in the cell culture as an indicator of stress. Samples from cell culture can be withdrawn and tested for GABA binding with GABA-A receptor concentrations using absorbance spectroscopy (see, for example, Elgarf et al., ACS Chem. Biol. 2018; 13, 8, 2033-2039, which is incorporated herein in its entirety), serotonin concentrations using ELISA (see, for example, Warkentin et al., ACS Chem. Biol. 2015 June; 90(6):564-72, which is incorporated herein in its entirety), dopamine concentrations using ELISA (see, for example, Yang and He, 2022, BMC Complement Med Ther. 2022 Mar. 3; 22(1):55, which is incorporated herein in its entirety), dopamine concentration using ELISA (see, for example, Nichkova et al., J Neurochem. 2013 June; 125(5):724-735, which is incorporated herein in its entirety), BDNF concentrations using ELISA (see, for example, Polacchini et al., Sci. Rep. 2015; 5:17989, which is incorporated herein in its entirety), cortisol concentrations using ELISA (see, for example, Yang and He, 2022, BMC Complement Med Ther. 2022 Mar. 3; 22(1):55, which is incorporated herein in its entirety), AEA concentrations using ELISA (see, for example, Buczynski and Parsons, Br J Pharmacol. 2010 June; 160(3):423-442, which is incorporated herein in its entirety), IL-6 concentrations using ELISA (see, for example, Chen et al., J Immunol Methods. 2016 July; 434:1-8., which is incorporated herein in its entirety), and IL-17 concentrations using ELISA (see, for example, Pappu and Dong, Curr Protoc Immunol. 2007 November; Chapter 6:6.25.1-6.25.8, which is incorporated herein in its entirety).

### Expected Results

Comparison of GABA binding with GABA-A receptor, serotonin, dopamine, BDNF, cortisol, AEA, IL-6, and IL-17 concentration levels in the cell culture with and without application of composition described herein will be performed to determine the efficacy of the composition in lowering stress.

### Example 3. Clinical Efficacy Testing of Composition on Reducing Oxidative Stress

### Protocol

Clinical efficacy studies are being conducted for the composition described herein using up to 100 subjects over a period of four (4) week. The clinical study protocol is described below.

### Study Group Selection

1. Inclusion Criteria
   a. Gender: Male and Female
   b. Non-smoking status
   c. Age: 18-55 years
   d. BMI: 24-40 kg/m²
   e. Maintenance of normal exercise and activity patterns
   f. Total cholesterol ≤7.758 mmol/L (300 mg/dl)
   g. Triglyceride concentration ≤3.387 mmol/L (300 mg/dl)
2. Exclusion criteria:
   a. Individuals with chronic illness such as high blood pressure, diabetes
   b. Individuals receiving prescription medication
   c. Individuals taking antioxidant supplements

### Study Type Selection

Placebo-controlled Randomized clinical study: Random allocation to either the group receiving the supplement under investigation or to a group receiving placebo treatment as the control

### Study Design Type

Parallel-group: Each participant is randomly assigned to a group, and all the participants in the group receive (or do not receive) composition described herein.

### Outcome Measurements

1. Will be based on plasma metabolites
2. Will include primary outcome and secondary outcome
3. Can be self-monitored questionnaire (or a smartphone app) or reported by people who know the individual participating in the study

### Results

Results obtained from the clinical study will be analyzed to determine efficacy of composition described herein using the following steps:
1. Perform appropriate statistical tests to estimate the change levels in the 95% confidence interval for the two study groups where the outcome measure is in the form of ordinal level scale. Examples of such test include:
   a. Wilcoxson Rank-Sum test
   b. Mann-Whiney U test
2. Perform an intention-to-treat (ITT) analysis to overcome the issue arising from dropouts i.e. “Attrition bias.”

### OTHER EMBODIMENTS

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

### BIBLIOGRAPHY

Chen F, Teachey D T, Pequignot E, Frey N, Porter D, Maude S L, Grupp S A, June C H, Melenhorst J J, Lacey S F. Measuring IL-6 and sIL-6R in serum from patients treated with tocilizumab and/or siltuximab following CAR T cell therapy. J Immunol Methods. 2016 July; 434:1-8.
Pappu B P, Dong C. Measurement of interleukin-17. Curr Protoc Immunol. 2007 November; Chapter 6:6.25.1-6.25.8. doi: 10.1002/0471142735.im0625s79. PMID: 18432994.
El-Farhan N, Rees D A, Evans C. Measuring cortisol in serum, urine and saliva—are our assays good enough? Ann Clin Biochem. 2017 May; 54(3):308-322
Nichkova, M., Wynveen, P. M., Marc, D. T., Huisman, H.; Kellermann, G. H. (2013). Validation of an ELISA for urinary dopamine: Applications in monitoring treatment of dopamine-related disorders. Journal of Neurochemistry, 125 (5), 724-735.
Polacchini A, Metelli G, Francavilla R, Baj G, Florean M, Mascaretti L G, Tongiorgi E. A method for reproducible measurements of serum BDNF: comparison of the performance of six commercial assays. Sci Rep. 2015 Dec. 10; 5:17989
Alshaimaa A. Elgarf, David C. B. Siebert, Friederike Steudle, Angelika Draxler, Guanguan Li, Shengming Huang, James M. Cook, Margot Ernst, and Petra Scholze. Different Benzodiazepines Bind with Distinct Binding Modes to GABAA Receptors. ACS Chemical Biology 2018 13 (8), 2033-2039.
Warkentin T E, Arnold D M, Nazi I, Kelton J G. The platelet serotonin-release assay. Am J Hematol. 2015 June; 90(6):564-72.
Ayyadurai V A S, Deonikar P, Fields C. Mechanistic Understanding of D-Glucaric Acid to Support Liver Detoxification Essential to Muscle Health Using a Computational Systems Biology Approach. Nutrients. 2023 Feb. 1; 15(3):733. doi: 10.3390/nu15030733. PMID: 36771439; PMCID: PMC9921405.
Ayyadurai, V. A. S.; Deonikar, P. In Silico Modeling and Quantification of Synergistic Effects of Multi-Combination Compounds: Case Study of the Attenuation of Joint Pain Using a Combination of Phytonutrients. Appl. Sci. 2022, 12, 10013.
Ayyadurai, V. A. S.; Deonikar, P.; Bannuru, R. R. Attenuation of low-grade chronic inflammation by phytonutrients: A computational systems biology analysis. Clin. Nutr. ESPEN 2022, 49, 425-435.
Ayyadurai, V. A. S., Prabhakar Deonikar, Kevin G. McLure, and Kathleen M. Sakamoto. 2022. “Molecular Systems Architecture of Interactome in the Acute Myeloid Leukemia Microenvironment” Cancers 14, no. 3: 756. https://doi.org/10.3390/cancers14030756
Ayyadurai, V. A. S.; Deonikar, P. Bioactive compounds in green tea may improve transplant tolerance: A computational systems biology analysis. Clin. Nutr. ESPEN 2021, 46, 439-452.
Ayyadurai, V. A. S., & Dewey, C. F. (2011). CytoSolve: A scalable computational method for dynamic integration of multiple molecular pathway models. Cellular and Molecular Bioengineering. https://doi.org/10.1007/s12195-010-0143-x
Ayyadurai, V. A. S., & Deonikar, P. (2015). Do GMOs Accumulate Formaldehyde and Disrupt Molecular Systems Equilibria? Systems Biology May Provide Answers. Agricultural Sciences. https://doi.org/10.4236/as.2015.67062
Koo, A., Nordsletten, D., Umeton, R., Yankama, B., Ayyadurai, S., García-Cardeña, G., & Dewey, C. F. (2013). In silico modeling of shear-stress-induced nitric oxide production in endothelial cells through systems biology. Biophysical Journal. https://doi.org/10.1016/j.bpj.2013.03.052
Sweeney, M. D., Ayyadurai, S., & Zlokovic, B. V. (2016). Pericytes of the neurovascular unit: Key functions and signaling pathways. Nature Neuroscience. https://doi.org/10.1038/nn.4288
Nordsletten, D. A., Yankama, B., Umeton, R., Ayyadurai, V. A. S., & Dewey, C. F. (2011). Multiscale mathematical modeling to support drug development. IEEE Transactions on Biomedical Engineering. https://doi.org/10.1109/TBME.2011.2173245
Ayyadurai, V. A. S. (2010). Services-Based Systems Architecture for Modeling the Whole Cell: A Distributed Collaborative Engineering Systems Approach. https://doi.org/10.1007/8754_2010_1
Ayyadurai. (2007) Scalable Computational Architecture for Integrating Biological Pathway Models (Doctoral Dissertation, Massachusetts Institute of Technology)
Subash-Babu P, Mohammed Alowaidh H, Al-Harbi L N, Shamlan G, Aloud A A, AlSedairy S A, Alshatwi A A. Ocimum basilicum L. Methanol Extract Enhances Mitochondrial Efficiency and Decreases Adipokine Levels in Maturing Adipocytes Which Regulate Macrophage Systemic Inflammation. Molecules. 2022 Feb. 18; 27(4):1388. doi: 10.3390/molecules27041388. PMID: 35209178; PMCID: PMC8876186.

1-19. (canceled)

20. A composition, comprising:
about 20% to about 40% w/w of kaempferol;
about 0.5% to about 10% w/w of hypericin;
about 2% to about 10% w/w of withanone;
about 10% to about 30% w/w of ursolic acid;
about 10% to about 30% w/w of oleanic acid; and
about 10% to about 30% w/w of catechin.

21. The composition of claim 20, comprising:
about 34.6% w/w of kaempferol;
about 1.05% w/w of hypericin;
about 3.35% w/w of withanone;
about 18.87% w/w of ursolic acid;
about 22.00% w/w of oleanic acid; and
about 20.13% w/w of Catechin.

22. The composition of claim 21, wherein the composition further comprises one or more excipients, diluents, or carriers.

23. The composition of claim 22, wherein the composition is a powder.

24. A method for improving the relaxation of a subject in need thereof, comprising: administering to the subject a composition of claim 20.

25. A method for improving relaxation in a subject, comprising: administering to the subject a composition of claim 21.

26. The method of claim 25, wherein the composition is administered orally.
